SEQUENCE LISTING (6 sequences)

SEQ ID NO: 1 - protein, 5 aa, Artificial Sequence
  Description of Artificial Sequence Synthetic peptide
CELYQ

SEQ ID NO: 2 - protein, 6 aa, Artificial Sequence
  Description of Artificial Sequence Synthetic peptide
CELYQK

SEQ ID NO: 3 - protein, 9 aa, Artificial Sequence
  Description of Artificial Sequence Synthetic peptide
ELYQKRVLT

SEQ ID NO: 4 - protein, 14 aa, Artificial Sequence
  Description of Artificial Sequence Synthetic peptide
EAEELYQKRVLTIT

SEQ ID NO: 5 - protein, 6 aa, Artificial Sequence
  Description of Artificial Sequence Synthetic peptide
EAETIT

SEQ ID NO: 6 - protein, 14 aa, Artificial Sequence
  Description of Artificial Sequence Synthetic peptide
EAEELYQGRVLTIT

(19) United States
(12) Patent Application Publication
(10) Pub. No.: US 2009/0170178 A1
(43) Pub. Date: Jul. 2, 2009

(54) Cell stimulating device and cell stimulating method

(75) Inventors: Miwako Ozaki, Tokyo (JP); Kouichi Itoh, Kagawa (JP)

Correspondence Address: GREENBLUM & BERNSTEIN, P.L.C., 1950 ROLAND CLARKE PLACE, RESTON, VA, 20191, US

(73) Assignee: RIKEN, Saitama (JP)

(21) Appl. No.: 10/553,601

(22) PCT Filed: Apr. 22, 2004
(86) PCT No.: PCT/JP04/05818

§ 371 Date: Jul. 23, 2007

(30) Foreign Application Priority Data
Apr. 22, 2003 (JP) 2003-116895

(51) Int. Cl. C12N 13/00; C12M 1/42

(52) U.S. Cl. 435/173.8; 435/289.1

(57) ABSTRACT

It is an object of the present invention to provide an electrical stimulating device for efficiently providing direct electrical stimulation to a large number of nerve cells in vitro without causing any injuries to such cells. The present invention provides a cell stimulating device which comprises: a first electrode serving as a positive or negative electrode that extends from one side of a culture vessel that is used for accommodating cultured cells to a point at which the first electrode is not in contact with the cultured cells or is in contact with the surfaces of the cultured cells; and a second electrode serving as a negative or positive electrode that extends from the other side of the culture vessel to a point at which the second electrode is not in contact with the cultured cells or is in contact with the surfaces of the cultured cells, wherein an electric field for stimulating cells is formed via the first electrode and the second electrode.

### TECHNICAL FIELD

The present invention relates to a cell stimulating device and a cell stimulating method. More specifically, the present invention relates to a cell stimulating device and a cell stimulating method whereby electrical stimulation can be applied to cultured cells in a state such that electrodes is not in contact with the cultured cells or is in light contact with the surfaces of the cultured cells.

### BACKGROUND ART

One of the features of nerve cells is that they have electrical activity, which is referred to as nervous activity. It is thought that, as immature nerve cells differentiate and develop, the nerve cells express ion channels, transmitter receptors, and the like in response to stimulation by neurotransmitters, neurotrophic factors, or the like, and thus they acquire the sensitivities to transmitters and excitability (inherent nervous activity) that are inherent to nerve cells, so that the thus obtained patterns show the history or individuality of each nerve cell. In recent years, it has been revealed that this type of nervous activity (namely, electrical stimulation) inversely controls behaviors of substances associated with a nerve.

The present inventors have conducted studies in order to clarify nervous activity and its biological functions. As a result, it has been revealed that there is a possibility that a pattern of nervous activity (a pattern of electrical activity) works as an information encoding system. Although the ultimate goal of the present inventors' research is to control brain plasticity by controlling the pattern of nervous activity, it is important in connection with such goal to clarify the roles of nervous activity patterns (neuronal impulses), and to decode and profile these patterns of nervous activity. For this purpose, it has been necessary to conduct experiments under artificially well-controlled stimulation conditions.

Further, the control of nerve cell electrical activity enables the control of brain plasticity, and is applicable for treatment. In the field of regenerative medicine, particularly with regard to nerve regeneration, it is considered to be essential to allow nerve cells to have access to electrical activity in order to acquire normal functions This requires an electrical stimulating device, whereby direct electrical stimulation can efficiently be applied to a large number of nerve cells in vitro without causing any injuries to such cell.

Known conventional methods for applying electrical stimulation to cells are: (1) a method wherein electrodes are inserted into nerve cells to stimulate them; and (2) a method wherein nerve cells are caused to adhere to a stimulation electrode base to receive stimulation. However, according to method (1), it is difficult to stimulate a large number of cells at the same time and also it has the drawback that it causes excessive damage to cells. Further, according to method (2), the number of nerve cells that can be caused to adhere to the electrode base is very small and therefore it is difficult to make a practical stimulating device that works according to this method.

### DISCLOSURE OF THE INVENTION

The object of the present invention is to solve the aforementioned problems of the prior art. That is, it is an object of the present invention to provide an electrical stimulating device for efficiently providing direct electrical stimulation to a large number of nerve cells in vitro without causing any injuries to such cells. It is another object of the present invention to provide a cell stimulating method whereby direct electrical stimulation can efficiently be applied to a large number of nerve cells in vitro without causing any injuries to such cells.

As a result of intensive studies to solve the above problems, the present inventors have found that desired electrical stimulation can be applied to cells in a manner such that:

a cell stimulating device is used, which comprises: a first electrode serving as a positive or negative electrode that extends from one side of a culture vessel that is used for accommodating cultured cells to a point at which the first electrode is not in contact with the cultured cells or is in contact with the surfaces of the cultured cells; and a. second electrode serving as a negative or positive electrode that extends from the other side of the culture vessel to a point at which the second electrode is not in contact with the cultured cells or is in contact with the surfaces of the cultured cells; and

an electric field is formed via the first electrode and the second electrode such that the cells are stimulated in the electric field. This has led to the completion of the present invention.

Specifically, in accordance with the present invention, a cell stimulating device is provided, such device comprising: a first electrode serving as a positive or negative electrode that extends from one side of a culture vessel that is used for accommodating cultured cells to a point at which the first electrode is not in contact with the cultured cells or is in contact with the surfaces of the cultured cells; and a second electrode serving as a negative or positive electrode that extends from the other side of the culture vessel to a point at which the second electrode is not in contact with the cultured cells or is in contact with the surfaces of the cultured cells, wherein an electric field for stimulating cells is formed via the first electrode and the second electrode.

Preferably, the aforementioned cell stimulating device is provided, such device comprising: a first electrode serving as a positive or negative electrode that extends from either a top or a side portion of a culture vessel that is used for accommodating cultured cells to a point at which the first electrode is not in contact with the cultured cells or is in contact with the surfaces of the cultured cells; and a second electrode serving as a negative or positive electrode that extends from the other top or side portion of the culture vessel to a point at which the second electrode is not in contact with the cultured cells or is in contact with the surfaces of the cultured cells, wherein an electric field for stimulating cells is formed via the first electrode and the second electrode.

Preferably, the aforementioned first electrode is a circular ring electrode.

Preferably, the aforementioned second electrode is a single-point electrode, a multi-point electrode (a plurality of electrodes), an electrode formed with mesh-sheet, or a sheet electrode comprising a multi-point electrode.

In another aspect of the present invention, a method for electrically stimulating cells with the use of any of the aforementioned cell stimulating devices, wherein an electric field is formed by the aforementioned first and second electrodes and cultured cells are stimulated in the electric field, is provided.

Preferably, the cultured cells that are stimulated by the method for electrically stimulating cells of the present invention are nerve cells.

### BRIEF DESCRIPTION OF THE DRAWINGS

FIG. 1 shows a cell stimulating device in which a first electrode comprises a circular ring electrode (small size) and a second electrode comprises a single-point electrode.

FIG. 2 shows a cell stimulating device in which a first electrode comprises a circular ring electrode (large size) and a second electrode comprises a single-point electrode.

FIG. 3 shows a cell stimulating device in which a first electrode comprises a circular ring electrode (large size) and a second electrode comprises a multi-point electrode (a plurality of electrodes).

FIG. 4 shows a cell stimulating device in which a first electrode is a circular ring electrode and a second electrode is a mesh-sheet electrode.

FIG. 5 shows a cell stimulating device in which a first electrode is a circular ring electrode and a second electrode is a sheet electrode comprising a multi-point electrode.

In FIGS. 1 to 5, a positive (or negative) electrode 1, a negative (or positive) electrode 2, wiring 3, a culture vessel 4, a culture vessel cover 5, cells 6, and a power source 7 are shown respectively.

FIG. 6 show proteolytic cleavage of neuregulin by PMA stimulation.

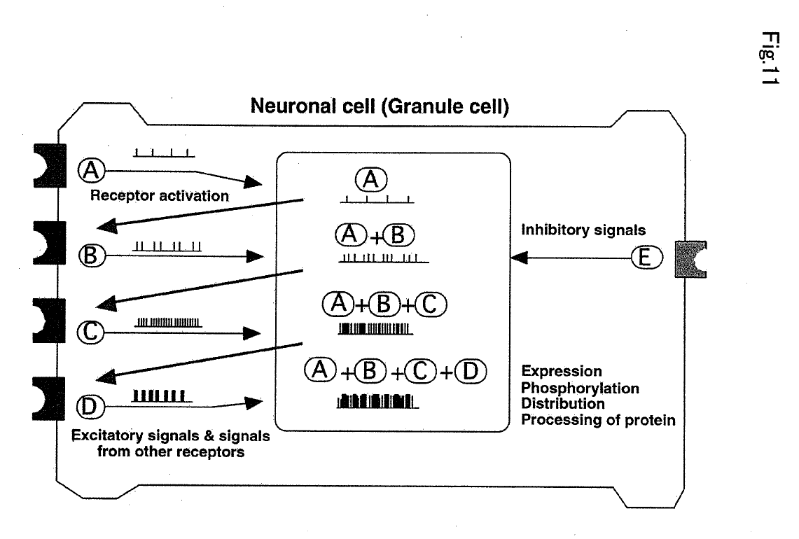

Pontine nuclei neurons and cerebellar granule cells were stained with an anti-NRGβ1 antibody for 7 days in vitro (7DIV). Both neurons were NRG-positive with respect to somatic and neuron processes (FIG. 6A) (scale bar: 60 μm).

In the case of FIG. 6B, pontine nuclei neurons and granule cells that had been transfected with transmembrane NRG and those that had not been transfected with the same were prepared, followed by PMA stimulation for 60 minutes. Then, the conditioned medium for each case was recovered. The tyrosine phosphorylation activity of each conditioned medium was examined with the use of cerebellar granule cells. A culture product of granule cells was stimulated for 5 to 10 minutes with each conditioned medium that had been recovered and concentrated. Lysates obtained from granule cells were degraded by SDS-PAGE. The blots were subjected to immunoprecipitation with an anti-ErbB4 antibody, followed by detection with an anti-phosphotyrosine antibody (4G10). 180-kD tyrosine-phosphorylated bands were detected by stimulation (fig. B). A conditioned medium was recovered from PN (non-transfected pontine nuclei neurons), tPN (transfected pontine nuclei neurons), GC (non-transfected granule cells), and tGC (transfected granule cells). Fig. C shows the summarized results. Experiments were independently repeated 3 or 4 times.

In the case of FIG. 6D, CREB phosphorylation following stimulation of each conditioned medium was confirmed using cerebellar granule cells. Serum-starved cerebellar granule cells were treated (for 5 to 10 minutes) with conditioned media that had been recovered from the culture products of pontine nuclei neurons and granule cells. The conditioned medium recovered from pontine nuclei neurons was used for stimulation in the cases of a, b, and c. In the cases of d, e, and f, a conditioned medium was prepared from the culture product of granule cells.

a; d: Control

b; e: Vector (pEGFP-N3)

c; f: pNRG-GFP

Stimulated granule cells were immobilized, followed by staining with a phospho-CREB antibody. The soluble forms that had been released after PMA stimulation (60 minutes) were concentrated and added to cultured granule cells. In panels c and f, the conditioned media should contain endogenous NRG and recombinant NRG. The difference between panels b and e indicates a function of NRG cleaved from recombinant mNRG. FIGS. 6E and 6F show the results of identification of an amino acid sequence that is necessary for proteolysis with the use of an ErbB and CREB phosphorylation assay system. The ELYQKRVLT sequence was located above extracellular portions of transmembrane domains that were aligned in parallel. When the sequence was subjected to deletion or mutation of lysine to glycine, proteolysis efficiency was inhibited as shown in FIG. 6F. The lysine residue was found to be an amino acid that is essential for recognition by a protease.

FIG. 7 shows CREB-phosphorylation activity induced by electrical stimulation.

Pontine neurons and cerebellar granule cells were prepared from a 18-day-old fetal mouse and a 7-day-old mouse. The neurons were cultured for 7 days so as to be subjected to different patterns of electrical stimulation. After electrical stimulation, CREB phosphorylation was examined using the granule cells (FIGS. 7A and 7B). The number of cells that were found to be positive for an anti-PCREB antibody was determined. The result was normalized relative to the total number of the cells. Images of 5 microscopic fields (×20) that had been randomly selected from each culture dish were taken for cell count. After independent experiments, 3 to 5 culture dishes were subjected to cell count. The highest efficiency of CREB phosphorylation was obtained upon stimulation at 50 Hz. Phosphorylation was partially blocked by TTX.

FIG. 8 shows proteolytic cleavage of NRG by electrical stimulation.

After electrical stimulation, tyrosine phosphorylation activity of ErbB4 was measured following PMA stimulation in a manner similar to that described above. Panel A shows that the highest efficiency of tyrosine phosphorylation activity in a conditioned medium for ErbB4 was obtained upon stimulation at 50 Hz in the cases of pontine nuclei neurons and granule cells. Tyrosine phosphorylation was blocked by H7 serving as a PKC inhibitor. The results are summarized in a graph (FIG. 8B). In the cases of FIGS. 8C and 8D, CREB phosphorylation was confirmed after conditioned medium stimulation with the use of cerebellar granule cells. Serum-starved cerebellar granule cells were tested (for 15 minutes) with a conditioned medium that had been recovered from a culture product of granule cells subjected to electric stimulation. Full-NRG: In this experiments, full-length NRG was transfected into granule cells. Del-NRG: Amino acid nos. 197 to 216 of NRG were deleted and thus the protein became resistant to proteolysis.

FIG. 8G shows the results of directly detecting the cleaved NRG by immunoblotting after immunoprecipitation. The detection technique is summarized in FIGS. 8E and 8F. A conditioned medium was recovered from approximately 5×107 granule cells into which MNRG had been transfected after electrical stimulation (transfection efficiency: not more than 5%). The medium was concentrated using a centricon, followed by immunoprecipitation with the use of an anti-sNRG antibody. The antibody used was an anti-sNRG polyclonal antibody (antibody produced in Example 1) that exclusively recognizes the C-terminal of the cleaved form of NRG

After immunoprecipitation, Western blot analysis was carried out using an anti-NRGβ1 antibody that exclusively recognizes NRG, 1. As shown in FIG. 8G a signal of the cleaved form of NRG was detected upon stimulation at 50 Hz. This signal disappeared by H7 serving as a PKC inhibitor. From these results, it is understood that the amount of released NRG in a medium varies depending on the frequency at which stimulation takes place.

FIG. 9 shows the expression of an NMDA receptor subunit and that of a GABAA receptor subunit, each of which was quantified by a real-time quantitative PCR method.

In the case of FIG. 9A, the expression of the NMDA receptor subunit and that of the GABAA receptor subunit were examined using granule cells that had been cultured it. vitro with the use of 10 mM KCl for 1 to 21 days. For an electrical stimulation experiment, 7DIV cells were selected. In the case of 7DIV, granule cells were still alive; however, the expression levels of NMDA receptor, NR2C, NR2B, and GABAA receptor β2 subunit mRNA had decreased. GABAA receptors α1 and γ2, MRNA were retained in the case of 7DIV. After different patterns of electrical stimulation, RT-PCR real-time quantitative analysis was carried out. Transcription of NR2C and that of β2 subunit were controlled at different frequencies. NR2C transcription was promoted upon stimulation at 1.0 and 100 Hz (FIG. 9B). The transcriptional increase detected at 100 Hz was blocked by TTX treatment. However, the increase at 1.0 Hz was not strongly blocked by TTX. Meanwhile, β2 transcription was strongly promoted upon stimulation at 0.1 to 10 Hz compared with NR2C. The increase was blocked by TTX treatment. The increase at 100 Hz was partially blocked by TTX. (0.1 Hz: n=6; 1 Hz: n=18; 10 Hz: n=10; 50 Hz: n=12; 100 Hz: n=26; No stimulation with TTX: n=3; 1 Hz and TTX: n=6; and 100 Hz and TTX: n=6; *p<0.001, **p<0.00001)

In the case of FIG. 9C, pharmacological experiments were carried out with electrical stimulation. In the case of NR2C, transcription, the level of which had increased at 1 and 100 Hz, was partially blocked by every antagonist and blocker. However, MK801 strongly blocked the expression of NR2 CmRNA. In the case of β2, in addition to MK801, CNQX blocked transcription upon stimulation at 1.0 and 100 Hz. The increase of mRNA at 100 Hz was strongly inhibited by a nonspecific calcium channel blocker. However, the increase at 1.0 Hz was not obviously blocked. In every case, receptor activation at a base level or higher was partially mimicked due to direct electrical stimulation (AP5: competitive NMDA receptor antagonist; MK801: noncompetitive NMDA receptor antagonist; CNQX: AMPA receptor antagonist; and Cd&EGTA: nonspecific calcium channel blocker). Each experiment was independently repeated 3 to 8 times.

FIG. 10 shows a schematic view of the expression of an NMDA receptor subunit. and that of a GABAA receptor subunit that are controlled in a frequency-dependent manner.

Cerebellar granule cells receive excitation signals from mossy fibers and inhibition signals from the Golgi apparatus via GABAA receptors. Neuronal activity patterns are determined based on the combination of such activities. Even granule cells that are not stimulated by mossy fibers have spontaneous activity. The expression of an NR2C subunit and that of a β2 subunit were detected at relatively low frequencies. However, the β2 expression was promoted to a greater extent than the NR2C expression (I). Meanwhile, the NR2C expression was more strongly induced at high frequencies (e.g. 100 Hz). There is a possibility that NR2C expression is induced by mossy fiber-stimulated granule cells, indicating that such expression is involved in considerable levels of receptor activation (III).

FIG. 11 shows models of receptor activation and patterns of neuronal activity.

### BEST MODE FOR CARRYING OUT THE INVENTION

The cell stimulating device of the present invention comprises: a first electrode serving as a positive or negative electrode that extends from one side of a culture vessel that is used for accommodating cultured cells to a point at which the first electrode is not in contact with the cultured cells or is in contact with the surfaces of the cultured cells; and a second electrode serving as a negative or positive electrode that extends from the other side of the culture vessel to a point at which the second electrode is not in contact with the cultured cells or is in contact with the surfaces of the cultured cells, wherein an electric field for stimulating cells is formed via the first electrode and the second electrode.

The aforementioned first and second electrodes constitute positive and negative electrodes, respectively, or vice versa. Thus, an electric field for stimulating cells is formed with the use of a combination of both electrodes. The aforementioned first and second electrodes are disposed in a manner such that each electrode extends from either a top or a side portion of a culture vessel to a point at which the electrode is not in contact with the cultured cells or is in contact with the surfaces of cultured cells.

In one embodiment of the present invention, the first electrode may be a circular ring electrode and the second electrode may be a single-point electrode, a multi-point electrode (a plurality of electrodes), a mesh-sheet electrode, or a sheet electrode comprising a multi-point electrode. Hereafter, the various aforementioned embodiments of the present invention are described based on the drawings.

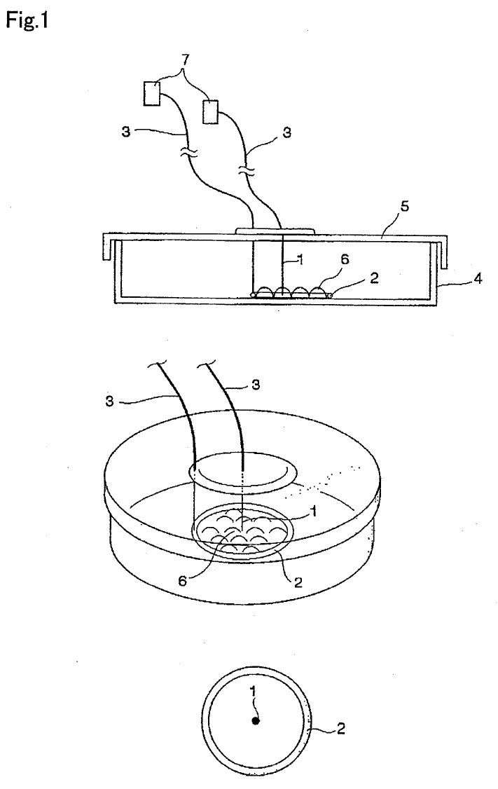

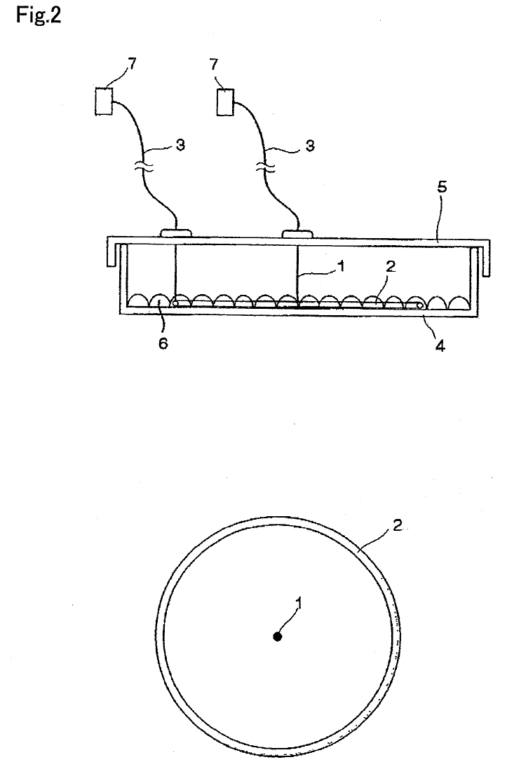

FIG. 1 shows a first embodiment of the cell stimulating device of the present invention. Specifically, FIG. 1 shows a cell stimulating device wherein a first electrode is a circular ring electrode (small size) and a second electrode is a single-point electrode. The upper drawing in FIG. 1 shows a side view of the device. The middle drawing in FIG. 1 shows a perspective view of the device. In both drawings, a positive (or negative) electrode 1 (single-point electrode) and a negative (or positive) electrode 2 (circular ring electrode) are each connected to a power source 7 via wiring 3, such wired electrodes passing through different points on a culture vessel cover 5. Further, each electrode extends to a point at which it comes into contact with the surfaces of cells 6 that are cultured on the bottom of a culture vessel 4. In addition, electrodes 1 and 2 are selected in a manner such that either one of them is a positive electrode and the other is a negative electrode. The bottom drawing in FIG. 1 shows a top view of the positional relationship of electrode 1 and electrode 2.

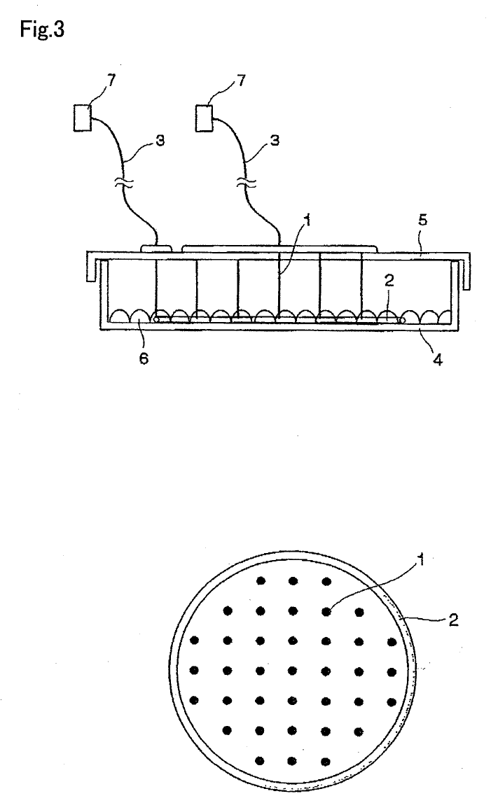

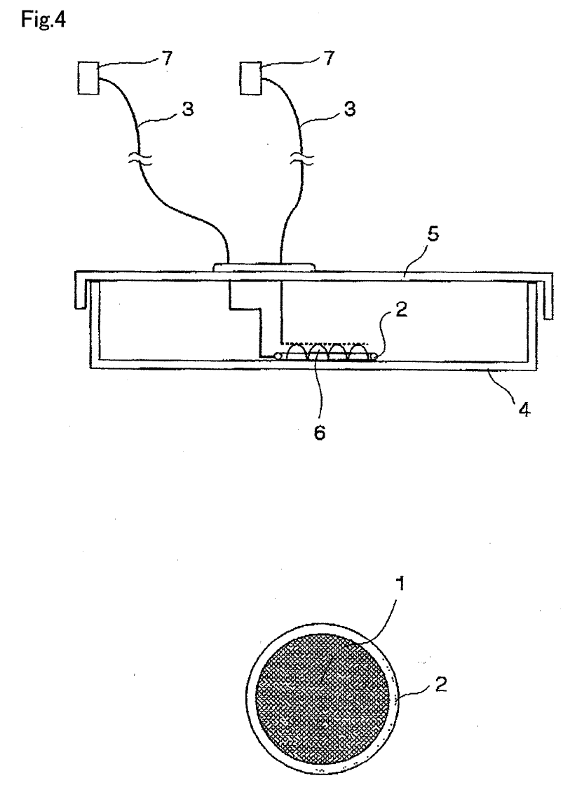

FIG. 2 shows a second embodiment of the cell stimulating device of the present invention. Specifically, FIG. 2 shows a cell stimulating device wherein a first electrode is a circular ring electrode (large size) and a second electrode is a single-point electrode. The upper drawing of FIG. 2 shows a side view of the device in which a positive (or negative) electrode 1 (single-point electrode) and a negative (or positive) electrode 2 (circular ring electrode) are each connected to a power source 7 via wiring 3, such wired electrodes passing through different points on a culture vessel cover 5. Further, each electrode extends to a point at which it comes into contact with the surfaces of cells 6 that are cultured on the bottom of a culture vessel 4. In addition, electrodes 1 and 2 are selected in a manner such that either one of them is a positive electrode and the other is a negative electrode. The bottom drawing in FIG. 2 shows a top view of the positional relationship of electrode 1 and electrode 2.

FIG. 3 shows a third embodiment of the cell stimulating device of the present invention. Specifically, FIG. 3 shows a cell stimulating device wherein a first electrode is a circular ring electrode (large size) and a second electrode is a multi-point electrode (a plurality of electrodes). The upper drawing of FIG. 3 shows a side view of the device in which a positive (or negative) multi-point electrode 1 (a plurality of electrodes) and a negative (or positive) electrode 2 (circular ring electrode) are each connected to a power source 7 via wiring 3, such wired electrodes passing through different points on a culture vessel cover 5. Further, each electrode extends to a point at which it comes into contact with the surfaces of cells 6 that are cultured on the bottom of a culture vessel 4. In addition, electrodes 1 and 2 are selected in a manner such that either one of them is a positive electrode and the other is a negative electrode. The bottom drawing in FIG. 3 shows a top view of the positional relationship of electrode 1 and electrode 2.

FIG. 4 shows a fourth embodiment of the cell stimulating device of the present invention. Specifically, FIG. 4 shows a cell stimulating device wherein a first electrode is a circular ring electrode and a second electrode is a mesh-sheet electrode. The upper drawing of FIG. 4 shows a side view of the device in which a positive (or negative) electrode 1 (mesh-sheet electrode) and a negative (or positive) electrode 2 (circular ring electrode) are each connected to a power source 7 via wiring 3, such wired electrodes passing through different points on a culture vessel cover 5. Further, each electrode extends to a point at which it comes into contact with the surfaces of cells 6 that are cultured on the bottom of a culture vessel 4. In addition, electrodes 1 and 2 are selected. in a manner such that either one of them is a positive electrode and the other is a negative electrode. In FIG. 4, the bottom drawing shows a top view of a positional relationship of electrode 1 and electrode 2. Within the second electrode 1, the electrode 2 is formed into a mesh and the electrode in its entirety constitutes a sheet.

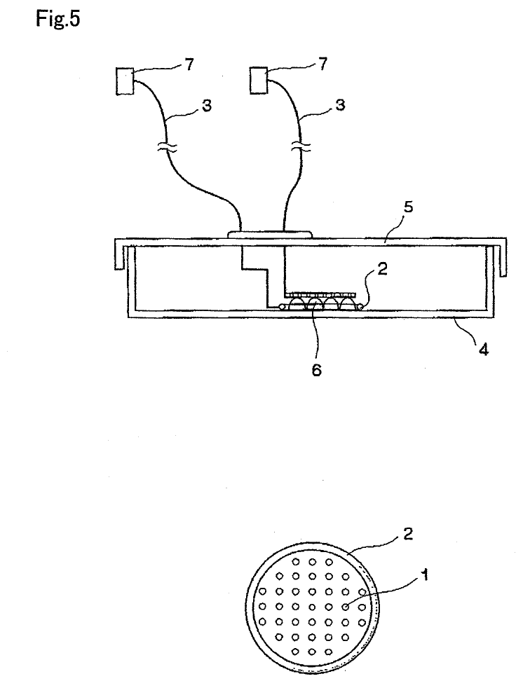

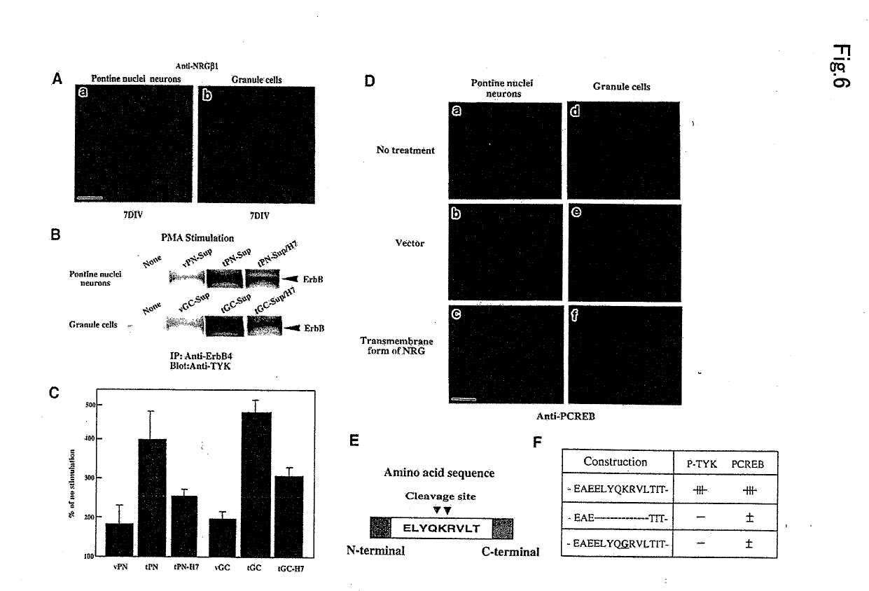

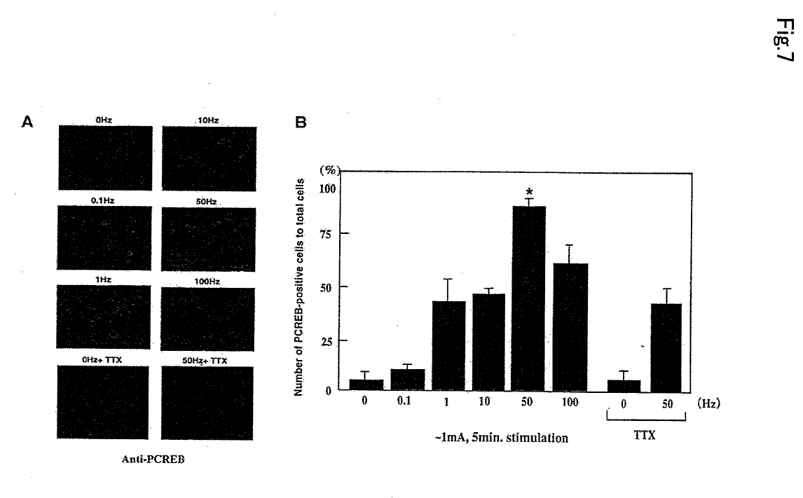

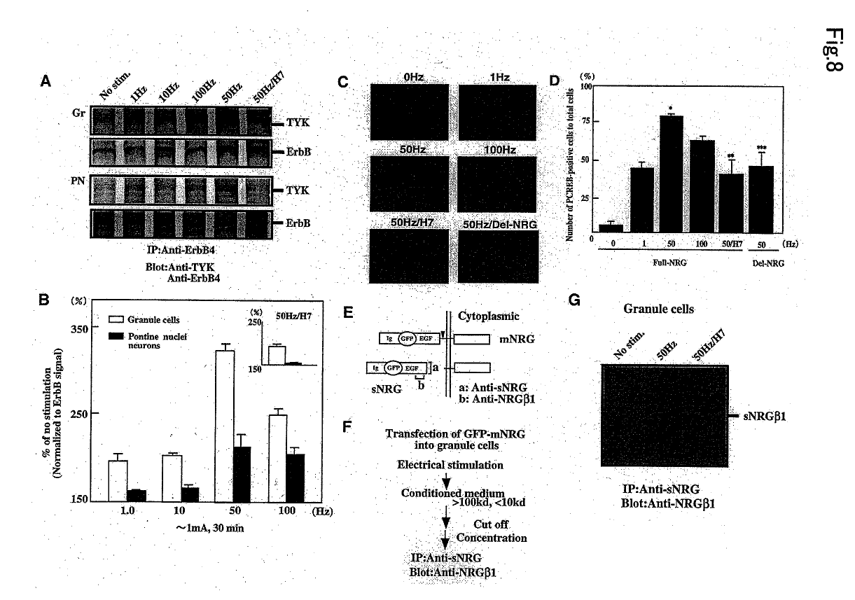

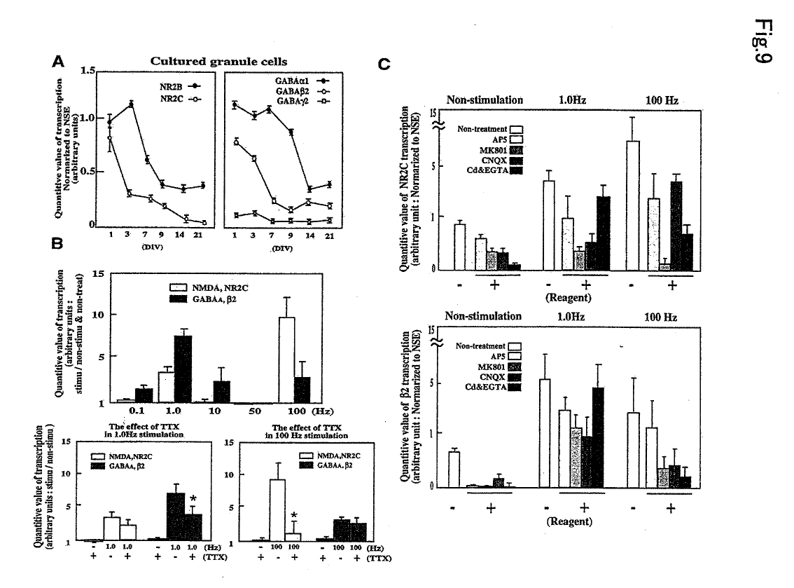

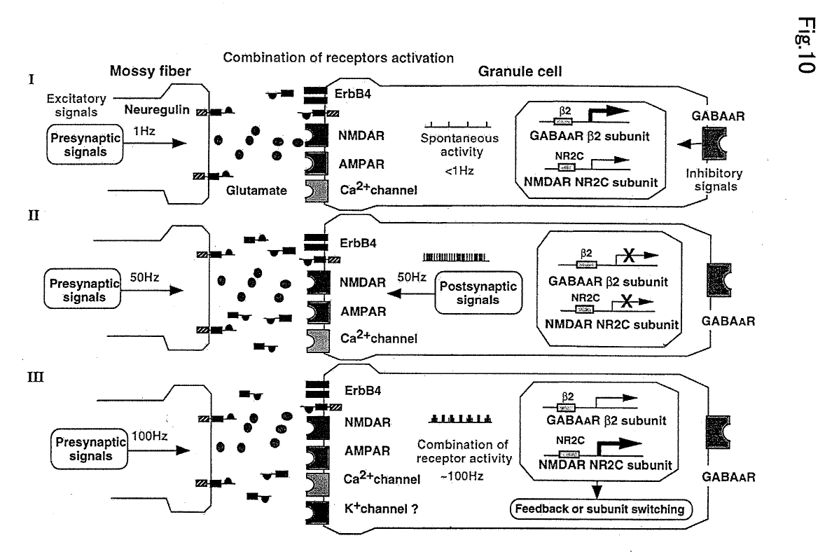

FIG. 5 shows a fifth embodiment of the cell stimulating device of the present invention. Specifically, FIG. 5 shows a cell stimulating device wherein a first electrode is a circular ring electrode and a second electrode is a sheet electrode comprising a multi-point electrode. The upper drawing of FIG. 5 shows a side view of the device in which a positive (or negative) electrode 1 (sheet electrode comprising a multi-point electrode) and a negative (or positive) electrode 2 (circular ring electrode) are each connected to a power source 7 via wiring 3, such wired electrodes passing through different points on a culture vessel cover 5. Further, each electrode extends to a point at which it comes into contact with the surfaces of cells 6 that are cultured on the bottom of a culture vessel 4. In addition, electrodes 1 and 2 are selected in a manner such that either one of them is a positive electrode and the other is a negative electrode. The bottom drawing of FIG. 5 shows a top view of a positional relationship of electrode 1 and electrode 2. In the second electrode 2, all of the many electrodes 1 (multi-point electrode) constitute a sheet.

In the cell stimulating device of the present invention, the structure of which may be that shown in any one of FIGS. 1 to 5, an electric field is formed in a medium contained in a culture vessel, such electric field being made up of a combination of positive and negative electrodes, each of which extends to a point at which it is not in contact with the cultured cells or is in contact with the surfaces of the cultured cells. In such electric field, it is possible to electrically stimulate cells. In accordance with the present invention, based on types or properties of cells to be stimulated, the intensity of an electric field to be formed, the region constituting the electric field, and the like, the most appropriate cell stimulating device can be adequately selected for use from among, the cell stimulating devices having the structures shown in FIGS. 1 to 5.

The present invention is hereafter described in greater detail with reference to the following examples, although the technical scope of the present invention is not limited thereto.

### EXAMPLES

### Example 1

### Preparation of Antibodies that Specifically Recognize Secretory Neuregulin

### (1) Design of Antigen Hapten Peptides

In order to prepare anti-peptide antibodies that complement a limited proteolytic reaction, information concerning the cleavage site of a target substrate protein is necessary. In this example, a peptide in which a cysteine residue is added to a short peptide (5 mer or 6 mer) containing the C-terminal of secretory neuregulin was synthesized, and was used as a hapten. To be more precise, a mixed peptide of Cys-Glu-Leu-Tyr-Gln and Cys-Glu-Leu-Tyr-Gln-Lys was used as an antigen.

### (2) Reagents Used:

Synthesized hapten peptide

KLH (keyhole limpet hemocyanin) in 50% glycerol (approximately 80% mg/ml) (Calbiochem)

DMFA (dimethylformamide)

MBS (m-maleimidobenzoyl-N-hydroxysuccinimide ester) (Pierce)

Gel filtration column (Pharmacia PD-10)

50 mM sodium phosphate buffer (pH 7.5)

100 mM sodium phosphate buffer (pH 7.2)

### Immunization:

Freund's complete adjuvant (FCA)

Freund's incomplete adjuvant (FIA)

Syringe, injection needle, and the like

### Affinity Purification of Antibodies;

100 mM HEPES buffer (pH 7.5)

Affigel 10 or 15 (BioRad)

30% acetic acid

20% ethanol

PBS

50 mM citrate buffer (pH 3.0)

2M TRIS buffer (pH 9.5)

20% glycerol-containing Na-PBS

### (3) Preparation of Antigens (Hapten/Carrier Complexes)

(i) MBS/activated KLH is prepared. Approximately 40 mg (0.5 ml) of KLH was added to 1.5 ml of 50 mM sodium phosphate buffer (pH 7.5), and the mixture is stirred by a stirrer. Then, a solution prepared by dissolving 9.3 mg of MBS into 0.38 ml of DMFA (to be prepared before use) is added thereto. After addition of MBS, the obtained solution is stirred at room temperature for 30 minutes. Thereafter, it was centrifuged at 2,000 rpm for approximately 2 minutes, and the supernatant was used below.
(ii) MBS/activated KLH is removed from free MBS. A Pharmacia PD-10 column is washed with 40 to 50 ml of 50 mM sodium phosphate buffer (pH 7.5) for equilibration. 2 ml of the supernatant obtained by centrifugation in process (i) is added to the column and infiltrated with a gel, and then 0.5 ml of the buffer is added thereto. After the infiltration is completed, eluate collection is started (first 2.5 ml of the eluate is discarded as prevoid), and 2 ml of the eluate (MBS/activated KLH) is collected. By this operation, a standard sample which can be used for 4 couplings can be obtained.
(iii) A synthesized hapten peptide is coupled with activated KLH. Approximately 5 mg of a synthesized peptide is dissolved into 4.5 ml of 100 mM sodium phosphate buffer (pH 7.2) and stirred. While dissolving, pH test paper is used to confirm that the pH of the solution is not reduced. 0.5 ml of MBS/activated KLH is added thereto, and the mixture is stirred at 4° C. for an entire day. It is not necessary to carry out dialysis after such process. The obtained products are used as antigens. They are preserved at −20° C. or −80° C.

### (4) Immunization

(i) Polyclonal antibodies were prepared using rabbits. Initially, a primary immunization was given to rabbits with individual weights of approximately 3 kg. 0.6 ml of FCA is added to 0.3 ml of an antigen solution, followed by stirring. Thereafter, the emulsion thereof is prepared by sonication [Branson Sonifier 185 (bath type), power 7 to 10, approximately 3 minutes]. The obtained emulsion is divided and injected into about 10 subcutaneous regions of right and left back muscles of each rabbit. 18G or 21G injection needles are used.
(ii) A second immunization is performed after approximately 1 month. In this case, emulsion is prepared in the same manner by adding 0.6 ml of FIA to 0.3 ml of the antigen solution. The obtained emulsion is injected into the right and left thigh muscles.
(iii) 2 weeks and 4 weeks after the second immunization, third and fourth immunizations are performed. In this case, 0.15 ml of antigen solution is diluted with 0.45 ml of PBS, and subcutaneous injection into the back is carried out in the same manner as in (i). 26G injection needles are used.
(iv) Approximately 1 week after the fourth immunization, partial blood collections are conducted. Approximately 40 to 50 ml of blood is collected and checked for antibody production, followed by affinity purification. When the results are favorable, 2 booster immunizations are performed after an approximately one-month rest, and then whole blood collection is conducted.

### (5) Affinity Purification of Antibodies

(i) Affinity gel is prepared. As an affinity carrier, Affigel 10 or 15 is used. First, 1 to 5 mg of hapten peptide is dissolved in 4 ml of 100 mM HEPES buffer (pH 7.5). Next, 1 to 2 ml of Affigel is washed on a glass filter by aspiration (twice with 10 ml of ice-cold distilled water) and then immediately added to the peptide solution. After rotating and stirring the obtained solution for an entire day at 4° C., aspiration washing is again conducted on a glass filter to remove free peptides. For this case, the obtained product is completely washed with 30% acetic acid or 20% ethanol in addition to a sufficient amount of distilled water, and is finally equilibrated with PBS. The obtained product is preserved in cold storage.
(ii) Specific antibodies are adsorbed onto an affinity column. Affinity gel is packed into a column (with an internal diameter of approximately 5 to 10 mm) and washed with PBS. 10 ml of inactivated serum is diluted with an equal amount of PBS, passed through a filter (0.22 or 0.45 μm), and then added to the column. Permeated liquid is collected, and the addition of the permeated liquid is repeated 3 to 4 times (at a flow rate of approximately 1 ml/minute). Further, the column is washed with approximately 50 ml of PBS.
(iii) Antibodies are collected. 0.5 ml of 2 M TRIS buffer (pH 9.5) is first placed into a tube, and antibodies that has been eluted from the affinity gel are collected into the tube. The elution is conducted by adding 5 ml of 50 mM citric acid buffer (pH 3.0) at a flow rate of approximately 1 ml/minute. Next, the eluate is transferred to a dialysis tube, followed by dialysis against Na-PBS containing 20% glycerol (for an entire day at 4° C.). The quantification of the antibodies is carried out by measuring absorption at 280 nm (1 mg IgG/ml, A₂₈₀=1.4). Usually, 1 to 10 mg of specific IgG is collected. Thereafter, the obtained antibodies are dispensed and preserved at −80° C.

### Example 2

### Pathological Action Mechanism of Neuregulin

### (Method)

### (1) Cell Preparation

Cerebellar granule cells and nerve cells of the pontine nuclei were prepared by the standard method from P7 and E18 BALB/c mice, respectively. 7 DIV cultures were used for PMA and electrical stimulation in both types of cells. The granule cells were cultured for 1 to 21 days in vitro under conditions including the presence of 10 mM KCl for quantification of receptor subunit expression. The pontine nuclei neurons were cultured with DMEM (Gibco BRL) containing 10% horse serum for 1 or 2 days. The neurons were then maintained with Neurobasal medium (Gibco BRL) supplemented with B-27 (Gibco BRL). The culture products were fed with a medium consisting of Neurobasal medium (Gibco BRL) supplemented with B-27 (Gibro BRL). The full length of the NRG plasmid containing the GFP tag and the GFP vector (pEGFP, Clonthech) were each transfected by Lipofectamine (TM) 2000 (Gibco BRL) (transfection efficiency of pontine nuclei neurons: 1 to 3%; granule cells: 5 to 10%). 24 to 36 hours after transfection, the neurons were stimulated with 1 μM PMA (Tocris) for 60 minutes.

### (2) Detection of Cleaved Form of NRG

The electrical stimulation in this Example was carried out using a cell stimulating device having a structure as described above and shown in one of FIGS. 1 to 5.

After electrical stimulation (1 mA, 30 to 60 V outside the cells) for 30 minutes of 5×10⁶ to 5×10⁷ cells transfected with recombinant full length mNRG, the conditioned media obtained from the cells were collected and concentrated using centricons 10 and 100 (Millipore) to remove large (>100 kd) and small (<10 kd) molecular weight proteins. The 7DIV granule cells were treated with the concentrated. conditioned media obtained from the pontine nuclei neurons and the granule cells. Upon the ErbB phosphorylation, Western blot analysis was performed in the standard method using a mouse monoclonal anti-phosphotyrosine antibody (4G10) after immunoprecipitation with a polyclonal anti-ErbB4 antibody (Santa Cruz) (Rieff H. I. et al., J Neurosci, 19(24), 10757-10766 (1999)). For immunoprecipitation studies, lysates were incubated with the appropriate diluted product of immunoprecipitating antibody for 1 hour at 4° C., followed by another 1-hour incubation at 4° C. with proteinA-Sepharose. Lysates were then centrifuged for 3 minutes at 15000 rpm, and the supernatant was discarded. The pellets were then washed twice with lysis buffer and were resuspended in gel loading buffer. Samples were boiled for 3 minutes, and proteins were separated by electrophoresis.

To detect CREB phosphorylation, 7DIV cultured granule cells were immobilized with 4% paraformaldehyde for 10 minutes after conditioned media stimulation for 10 to 15 minutes. Then, the immobilized granule cells were stained with a polyclonal anti-NRGβ1 antibody and a polyclonal anti-PCREB antibody (BioLabs). The stained granule cells were observed with a laser confocal microscope (Carl Zeiss). Alexa (TM) dyes (Molecular probe) were used as a secondary antibody.

### (Results)

### (1) Proteolytic Cleavage of Transmembrane NRG in Pontine Nuclei Neurons and Cerebellar Granule Cells

The condition of dissociated primary culture products of cerebellar granule cells and pontine nuclei neurons used for the experiments of FIG. 6A was investigated. Pontine nuclei neurons (PN) were prepared from embryonic day 18 (E18), and cerebellar granule cells (GC) were prepared from postnatal day 7 (P7). In order to examine whether or not proteolytic cleavage of mNRG occurs in cultured neurons, immunoprecipitation with anti-ErbB and anti-phosphotyrosine antibodies and immunocytochemical analyses using anti-PCREB antibody were performed with the use of pontine nuclei neurons and granule cells, which were transfected with or without recombinant full-length NRGβ1 containing a GFP tag and then stimulated with the PKC activator, namely phorbol-12-myristate-13-acetate (PMA), for 60 minutes.

With regard to the NRG receptor in the synapses between mossy fibers and granule cells, it is previously reported that ErbB2 and ErbB4 are involved in the cerebellar system (Ozaki M. et al., Nature, 390, 691-694 (1997); and Ozaki M. et al., Neurosci Res, 30 (4), 351-354 (1998)). The expression of ErbB4 was more strongly observed in cerebellar granule cells in vitro and in vivo than ErbB2. Phosphorylation of cyclic AMP responsive element binding protein (CREB) was involved in further downstream of the ErbB4 signal transduction pathway (Taberbero A. et al., Mol Cell Neurosci, 10, 309-322 (1998)). Conditioned media of the culture products of pontine nuclei neurons and granule cells after treatment with PMA were collected, concentrated, and applied to granule cells for 5 to 10 minutes. The lysates from granule cells were analyzed by SDS-PAGE after immunoprecipitation with an anti-ErbB4 antibody, and the resulting blot was detected with an anti-phosphotyrosine antibody (anti-TYK). Conditioned media were collected from non-transfected pontine nuclei neurons (None), vector-transfected pontine nuclei (vPN), NRG-transfected pontine nuclei neurons (tPN), vector-transfected granule cells (vGC), and mNRG-transfected granule cells (tGC). Both the mNRG-transfected pontine nuclei neurons and granule cells exhibited stronger phosphorylation activity than non-transfected neurons and vector-only-transfected cells (see FIGS. 6B and 6C). The amount of increased sNRG was confirmed by ErbB phosphorylation with the use of granule cells. Tyrosine-phosphorylated bands of 180 kD derived from conditioned media obtained from mNRG-transfected neurons were clearly observed when using PMA stimulation (FIG. 6B). The phosphorylation activity was suppressed by the conditioned media when the transfected pontine nuclei neurons and granule cells were treated with PKC inhibitor H7. Endogenous NRG did not. exhibit any obvious activity of ErbB phosphorylation. However, when recombinant mNRG was transfected into the cultured neurons, ErbB4 phosphorylation was clearly observed. These results indicate that sNRG was produced from recombinant mNRG following PKC activation. In FIG. 6C, the ratio of phosphorylation was normalized against ErbB4 signal which was blotted after immunoprecipitation with an anti-ErbB4 antibody.

The results of CREB phosphorylation are shown in FIG. 6D. The soluble forms released by PMA stimulation (60 minutes) were concentrated using a spin column and added into the cultured granule cells. The stimulated granule cells were stained with an anti-phospho-CREB antibody after fixation. The conditioned medium derived from the pontine nuclei neurons was used for stimulation (a, b and c) and the conditioned medium derived from granule cell cultures was used for stimulation (d, e and f). Panel D shows controls (a, d), vectors (b, e), and full-length NRG (c, f). The conditioned media (c and f) should have contained cleaved endogenous and recombinant NRG. The difference between b and c and that between e and f indicate CREB phosphorylation induced by sNRG derived from recombinant mNRG. Different types of CREB phosphorylation were observed after 5 or more minutes of treatment with the conditioned medium. An obvious release of NRG by KC1 stimulation was not observed when living cultured granule cells were used.

The conditioned media obtained from pontine nuclei neurons and granule cells transfected with full-length mNRG showed different ErbB- and CREB-phosphorylation activities. From measurement of ErbB- and CREB-phosphorylation activities, amino acid sequences necessary for proteolytic cleavage were identified. As shown in FIGS. 6E and 6F, deletion mutants inside the ELYQKRVLT region did not represent a clear proteolytic cleavage. Point mutation from K to G within this region also caused reduced cleavage as shown in the table and FIG. 6F. NRG has been reported as a substrate of the metalloprotease (ADAMs) family protease (Shirakabe K. et al., J Biol Chem, 276 (12), 9352-9358 (2000)). NRG cleavage by metalloprotease has been reported to occur mainly in the Golgi apparatus. One form of proteolytic cleavage of mNRG has already been reported to occur on the cell surface (Loeb J A. et al., Mol. Cell Neurosci, 11 (1-2), 77-91 (1998)). The proteolytic cleavage of NRG may be regulated by several proteases, depending on cell type, the protein localization in the cases of NRG and protease, and timing.

### (2) Proteolytic Cleavage of NRG by Patterned Electrical Stimulation

CREB-phosphorylation activity was measured by immunocytochemistry with the use of an anti-PCREB antibody to examine the optimum condition for CREB phosphorylation of cerebellar granule cells upon electrical stimulation. The granule cells were directly electrically stimulated at different frequencies for 5 minutes (see FIGS. 7A and 7B). The phosphorylation activity was observed at frequencies from 1 Hz to 100 Hz. The optimum frequency was found to be 50 Hz. The CREB-phosphorylation activity at 50 Hz was blocked by the sodium channel blocker TIX, resulting in 36.6±5.45% of the activity. In FIG. 8B, the PCREB-positive cells were counted and normalized against total cell numbers. These experiments suggest that different frequencies cause production of different conditions inside neuronal cells. The optimum frequency to cause proteolytic cleavage of NRG was 50 Hz.

In order to ascertain whether the proteolytic cleavage of NRG occurs with different patterns of electrical stimulation, ErbB phosphorylation was detected by immunoprecipitation with the use of anti-TYK and anti-ErbB4 antibodies after electrical stimulation at different frequencies. Culture products of GC and PNN were electrically stimulated in a 35-mm dish equipped with multiplatinum electrodes that were connected in parallel to a stimulating device. Electrical stimulation was carried out by continuously applying a steady-state current at 0.1 to 100 Hz for 30 seconds with the use of 1-mA alternating current in the form of a 0.2-msec rectangular pulse. The neurons. were stimulated at different frequencies for 30 minutes such that a sufficient cleaved form of NRG was obtained. Calcium levels at different steady states were separately imparted to the neurons that had been stimulated in different manners. A high frequency corresponds to a calcium level at a high steady state. The pH and the temperature of a medium during stimulation were the same as those in the case of no stimulation.

The cleaved form of NRG precipitated with anti-ErbB antibody was detected by immunobloting with the use of an anti-TYK antibody. The efficiency of phosphorylation was measured by normalization against ErbB4 signals. The phosphorylation signal was significantly stronger at 50 Hz of stimulation than at any other frequency of stimulation (FIGS. 8A and 8B). CREB-phosphorylation activities of the conditioned media also showed an optimal value at 50 Hz of stimulation (FIGS. 8C and 8D). Further, upon CREB phosphorylation, the conditioned medium derived from PNN showed a tendency similar to that observed in the case of GC. With stimulation at 50 Hz, 77.4±2.08% GCs were PCREB-positive. With stimulation at 100 Hz, 62.5+4.17% GCs were PCREB-positive. A reaction peak of PCREB was observed at 50 Hz (n=15, P<0.00018 against control, t test). CREB phosphorylation induced by stimulation at 50 Hz was inhibited by H7 (n=7, P=0.003 against stimulation at 50 Hz). When granule cells were transfected with non-cleaved NRGs (deleted mutants) and the cells were then stimulated at 50 Hz, the level of pCREB was significantly reduced (n=5, P=0.005 against stimulation at 50 Hz).

After using the above methods, the cleaved form of NRG was detected using the procedures described in FIGS. 8E to 8G An antibody used was the antibody (anti-sNRG antibody) produced in Example 1, such antibody being capable of exclusively recognizing the C-terminal of the cleaved form of neuregulin. Approximately 5×10⁷ transfected granule cells were used to collect conditioned media after electrical stimulation. Proteins of not less than 100 kD and of less than 10 kD were removed using centricons 10 and 100 by centrifuge filtration, followed by further concentration using a centricon 10. Thereafter, immunoprecipitation was performed with the use of an anti-sNRG antibody, followed by immunoblotting with an anti-NRGβ1 antibody capable of exclusively recognizing the β1 isoform of NRG. The result of blotting is shown in FIG. 8G. The signal of the cleaved NRG was observed at a position of approximately 30 to 40 kD. In the case of 50 Hz stimulation with H7, no obvious signal of the cleaved form of NRG was detected. As a result, it was revealed that sNRG is produced from mNRG via proteolytic cleavage that is initiated and controlled by specific patterns of electrical stimulation.

### Example 3

### Analysis of Expression Mechanism of Transmission Receptor

### (Method) Real-Time Quantitative Analysis of NMDA and GABAA Receptor Subunits

After electrical stimulation, a real-time quantitative analysis (ABI prism 7700, Perkin Elmer) was performed. Primers and TaqMan probes were designed using Primer Express (PE Biosystems). Each PCR product amplified by a different primer was found as a single band on agarose gel. The products were confirmed by direct sequencing. No primer used crossed with any other genes. For the pharmacological experiments, TTX (1 μM, Tocris), D-AP5 (50 μM, Tocris), MK801 (25 μM, Tocris), CNQX (10 μM, Tocris), Cd (100 μM, Wako Inc.), and EGTA (1 mM, Sigma) were used.

### (Results) The Expression of NMDA and GABAA Receptor Subunits Controlled by Electrical Stimulation

The patterns of electrical activities whereby the expression of an NMDA receptor subunit and that of a GABAA receptor subunit can be controlled were examined. With the use of a real-time quantitative polymerase chain reaction (PCR) method, the mRNA expression of an NMDA receptor subunit and that of GABAA receptor subunit were quantified during culture from 1 to 21 days in vitro. First, the mRNA expression level of each subunit of the NMDA and GABAA receptors in cultured granule cells was examined (FIG. 9A). Neural specific enolase (NSE) was used as a control. The properties of 7DIV cultured neurons (days in vitro) prepared from P7 mice are theoretically identical to those of P14 mice in vivo during a maturation period. In the case of P14 in vivo, NR2B expression was shut down in cerebellar granule cells, while on the other hand, NR2C expression was observed in all granule cells. The subunit switching of the NMDA receptor was almost completed in the cases of the P14 mice. On the other hand, α1, β2, and γ2 subunits of GABAA were abundantly expressed in the case of P14 in vivo. In order to adjust in vitro conditions to match in vivo conditions, granule cells that had been cultured for 7 days in vitro were chosen for electrical stimulation.

The cultured granule cells were stimulated at various frequencies (0 to 100 Hz) of 1 mA for 30 minutes (FIGS. 9B and 9C). Although the cell biological activity after stimulation was confirmed by chemical staining and anti-NRG antibody staining, no significant difference in the number of living cells was observed in any case. In the cases of the NMDA receptor NR2B and GABAAα2, γ1, no obvious effect of electrical stimulation was observed at any frequency. The expression of the NMDA receptor NR2C subunit was promoted by direct stimulation at frequencies of 1 and 100 Hz, and the increase found in the case of high frequency stimulation such as 100 Hz was blocked by TTX. The increased level of NR2C expression due to stimulation at 1 Hz was not strongly blocked by TTX. In the case of the GABAA receptor β2 subunit, the mRNA expression increased with stimulation at a low frequency such as 0.1 to 10 Hz. The increase with low frequency stimulation was partially blocked by TTX. However, the β2-increase at 100 Hz was not blocked by TTX.

Pharmacological experiments showed that the activities of NMDA and AMPA receptors and calcium channels were involved in retaining of NR2C expression and β2 expression. In the case of NR2C subjected to 1 Hz stimulation, the mRNA expression was strongly inhibited by NMDA and AMPA receptor antagonists. At a stimulation frequency of 100 Hz, MK801 (a non-competitive NMDA receptor antagonist) blocked NR2C elevation in a particularly strong manner. In addition, calcium channels contributed to NR2C expression rather than that of the AMPA receptor. In the case of β2 subjected to 1 Hz stimulation, the result was similar to that of NR2C subjected to stimulation at a frequency of 1 Hz. The MRNA increase in the case of β2 subjected to 100 Hz stimulation was inhibited by NMDA and AMPA receptor antagonists and a calcium channel blocker (non-specific blocker; Cd & EGTA). In the cases of both NR2C and β2, the calcium channel blocker strongly inhibited the subunit expression at a high frequency, but it did not inhibit the same at a low frequency. It is understood that the combination of the granule cell receptors involved and the degree of activity are controlled by different frequencies. Moreover, with a specific level of electrical stimulation, it was possible to partially restore the normal activity even in the presence of the antagonists and the blockers. An example in which the calcium channel blocker was used for stimulation at 1 Hz is shown (FIG. 9C, case of β2).

### (C) Discussion

### (Discussion of Examples)

As shown in FIG. 10, cerebellar granule cells are thought to receive a balance of excitatory and inhibitory signal input from mossy fibers and Golgi cells via NMDA and GABAA receptors. Patterns of neuronal firing in granule cells vary during synaptic development due to the involvement of various receptors. Patterns of the final neural activity in granule cells are most likely to be determined by a combination of molecules such as transmitters, neuropeptides, neurotrophic factors, and others related to environmental stimulation, including presynaptic neurons. Different combinations of molecules should result in different patterns of neuronal firing based on the relationship between molecular behaviors and patterns of electrical activity. Some forms of gene. expression have been reported to be regulated by patterned electrical activities (Buonanno A. et al., Curr Opin Neurobiol, 9, 110-120 (1999)). It is certain that molecular phosphorylation activity is controlled by patterned electrical activity (Buonanno A. et al., Curr Opin Neurobiol, 9, 110-120 (1999)).

In Example 2, it is proved that protein processing such as proteolytic cleavage is controlled by patterned electrical activity. The proteolytic cleavage of NRG was detected from low to high frequencies. However, a stimulation frequency at 50 Hz was the most appropriate for proteolytic cleavage of NRG from both mossy fibers (presynaptic cells) and granule cells (postsynaptic cells). This phenomenon supports the existence of a mechanism in which patterns of nerve activity are synchronized between presynaptic and postsynaptic cells, from a molecular viewpoint. Presynaptic signals first activate postsynaptic neurons, and then they synchronize presynaptic and postsynaptic neurons. When postsynaptic cells synchronize with presynaptic cells, the postsynaptic neurons may autoactivate with an autocrine mechanism so as to enter stage III (FIG. 10). Another possibility is that the mNRG may undergo stimulation-dependent proteolytic cleavage after the exchange of molecular information through a signal from the presynaptic neuron and bi-directional signal transduction over the course of synaptic formation. In either case, 50 Hz stimulation is thought to correspond to the intermediate stage of transduction between pre- and post-synaptic neurons (FIG. 10, II).

Furthermore, the molecular mechanism for NR2C expression and β2 expression, which are regulated by sNRG, has been revealed. With low-frequency stimulation (1 Hz), the amount of β2RNA transcribed was much greater than that of NR2C RNA throughout the activation of glutamate and ErbB receptors. To a greater-extent than the case of β2, NR2C mRNA induction was intensified at a high frequency (100 Hz), accompanied by glutamate receptor (particularly NMDA receptor) activation. The expression of an NR2C subunit and that of a β2, subunit were not observed at 50 Hz, which was the optimal frequency for proteolytic cleavage of NRG. It has already been proposed that NR2C expression requires neural activity and that the production efficiency of soluble forms of NRG might be controlled by electrical activity (Ozaki M. et al., The Neuroscientist, 7(2), 146-154 (2001)). In this Example, it was demonstrated for the first time that proteolytic cleavage of NRG is controlled by patterns of electrical activity in a frequency-dependent manner. Although NRG is necessary to induce NR2C subunit expression and β2 subunit expression, discrepancy in optimal value of frequency arose between the expression stage (FIG. 10, I and III) and the intermediate stage (FIG. 10, II). Pharmacological experiments were performed in order to explain such discrepancy. A plurality of receptors, such as the ErbB receptor, were found to be involved in controlling NR2C subunit expression and β2 subunit expression based on the results of the pharmacological experiments.

The experiments of direct electrical stimulation suggest the following two matters: (1) endogenous nerve activity is required to receptor activation that is necessary for inducing gene expression; and (2) direct electrical stimulation can partially compensate for the effects of receptors and ion channel blockers (see FIG. 9C). Over the course of synaptic maturation, there may be a cascade between specific patterns of neuronal activity and receptor activation (FIG. 11). When a receptor A is activated, neurons have a pattern A of neuronal activity. Subsequently, a receptor B is activated by the pattern A such that a pattern B is generated. As a result, neurons have an activation pattern that is a combination of pattern A and pattern B. Each pattern of activity in the cascade may control molecular behaviors. It is thought that specific patterns control molecular behaviors and that the patterns of neuronal firing are composed of a combination of activations of individual receptors or channels. Each composed pattern should contain a certain process for controlling molecular behaviors such as processing of a gene-expressing phosphorylated protein. Accordingly, the combination of activated receptors and ion channels and the order of activation of such receptors and channels may serve as keys to clarify the discrepancy mentioned above.

In addition, it was possible to compensate for some effects of the receptors that can be blocked by antagonists or blockers with specific levels of electrical stimulation. This means that the role of presynaptic neurons, receptors, and channel activity can be mimicked with a. specific pattern of electrical activity. Thus, it is important to examine the role of patterns of electrical activity for artificially controlling neuron formation.

### INDUSTRIAL APPLICABILITY

In accordance with the present invention, electrical stimulation is not directly applied to cells, resulting in reduced damage to cells. In addition, electrical stimulation can be applied to many cells at once. Further, since cells are placed on a path through which electric current flows, electrical stimulation can efficiently be applied to cells over a short period of time. That is, in accordance with the present invention, electrical stimulation can be directly applied in vitro to many nerve cells with good efficiency without causing damage.

1. A cell stimulating device which comprises: a first electrode serving as a positive or negative electrode that extends from one side of a culture vessel that is used for accommodating cultured cells to a point at which the first electrode is not in contact with the cultured cells or is in contact with the surfaces of the cultured cells; and a second electrode serving as a negative or positive electrode that extends from the other side of the culture vessel to a point at which the second electrode is not in contact with the cultured cells or is in contact with the surfaces of the cultured cells, wherein an electric field for stimulating cells is formed via the first electrode and the second electrode.

2. The cell stimulating device according to claim 1 which comprises: a first electrode serving as a positive or negative electrode that extends from either a top or a side portion of a culture vessel that is used for accommodating cultured cells to a point at which the first electrode is not in contact with the cultured cells or is in contact with the surfaces of the cultured cells; and a second electrode serving as a negative or positive electrode that extends from the other top or side portion of the culture vessel to a point at which the second electrode is not in contact with the cultured cells or is in contact with the surfaces of the cultured cells, wherein an electric field for stimulating cells is formed via the first electrode and the second electrode.

3. The cell stimulating device according to claim 1 wherein said first electrode is a circular ring electrode.

4. The cell stimulating device according to claim 1 wherein said second electrode is a single-point electrode.

5. The cell stimulating device according to claim 1 wherein said second electrode is a multi-point electrode that is a plurality of electrodes.

6. The cell stimulating device according to claim 1 wherein said second electrode is an electrode formed with mesh-sheet.

7. The cell stimulating device according to claim 1 wherein said second electrode is a sheet electrode comprising a multi-point electrode.

8. A method for electrically stimulating cells with the use of the cell stimulating device of claim 1, wherein an electric field is formed by said first and second electrodes and cultured cells are stimulated in the electric field.

9. The method for electrically stimulating cells according to claim 8 wherein the cultured cells are nerve cells.
